(19) United States
(12) Patent Application Publication
(10) Pub. No.: US 2001/0049518 A1
(43) Pub. Date: Dec. 6, 2001

(54) Method of delivering a beneficial agent employing a steerable catheter

(76) Inventor: Reuben M. Hoch, Boca Raton, FL (US)

Correspondence Address: GUNSTER, YOAKLEY & STEWART, PA, BROWARD FINANCIAL CENTRE, SUITE 1400, 500 EAST BROWARD BLVD, FT LAUDERDALE, FL, 33394, US

(21) Appl. No.: 09/797,423

(22) Filed: Mar. 1, 2001

Related U.S. Application Data
(60) Provisional application No. 60/186,042, filed on Mar. 1, 2000.

(51) Int. Cl. A61M 37/00; A61M 31/00

(52) U.S. Cl. 604/510; 604/95.04

(57) ABSTRACT

A method of delivering a beneficial agent is provided that employs a steerable catheter to precisely locate and deliver a beneficial agent such as a pain medication to a selected site in a patient. The method includes maneuvering the catheter under fluoroscopic observation to specific sites such as a nerve root to control pain.

### CROSS-REFERENCE TO RELATED APPLICATION

This application is related to and claims priority from U.S. Provisional Patent Application Ser. No. 60/186,042, filed Mar. 1, 2000, entitled DIRECTIONAL PAIN INTERVENTION CATHETER, the entirety of which is incorporated herein by reference.

### FIELD OF THE INVENTION

The present invention relates to a method of delivering a beneficial agent to a patient, and in particular, to a method of delivering a pain medication to a precisely determined location within an epidural space employing a steerable catheter.

### BACKGROUND OF THE INVENTION

Pain and pain management is a necessary focus of modern medicine. Patients have come to expect that medical science can at least relieve pain, even if a cure is currently unavailable.

One of the most common pain complaints is associated with the back. The back is a complex structure that includes the spinal column, muscles, nerves and associated blood and lymphatic networks. The spinal column is made up of individual vertebrae and intervertebral discs between the vertebrae. Enclosed within the spinal column are nerves which are surrounded by the dura. Extending the length of the spine is the epidural space which contacts the dura. It is known to inject pain medications within the epidural space to control back pain. The epidural space contains fat, connective tissue, blood vessels, nerves and other structures. It is known in the art to deliver medications to various locations within the epidural space; however, due to the complexity of the space, it is difficult to deliver the medications to a precise location, such as a specific nerve root. Current methods of delivering medications rely on the medications diffusing or percolating through the epidural space or techniques that deliver medications to the subarachnoid space but it is difficult to correctly position the catheter to deliver medicaments in the most effective manner using the lowest dosage possible.

It is desirable to have a method of delivering medicaments to precise locations within a patient's epidural space.

### SUMMARY OF THE INVENTION

The present invention provides a method of delivering a beneficial agent to a patient employing a steerable catheter comprising the steps of: radiologically positioning the steerable catheter within a selected target area of the patient to be treated; imaging the steerable catheter wherein an operator is able to precisely maneuver the catheter to a specific location within the patient; and injecting the beneficial agent at the specific location through the steerable catheter.

The present invention also provides a method of delivering medication employing a steerable catheter comprising the steps of: placing a patient in a prone position; examining a target area of the patient; marking an entry location within the target area; sterilizing the target area; inserting an introducer needle into the target area at the entry location; confirming that the introducer needle is correctly positioned within the target area; inserting the steerable catheter into the introducer needle; maneuvering the steerable catheter to a precisely determined location within the target area; confirming that the steerable catheter is correctly positioned at the precisely determined location radiologically; and injecting the medication through the steerable catheter wherein the medication is delivered to the precisely determined location.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates a catheter used in accordance with the invention;

FIG. 2 illustrates an injection hub;

FIG. 3 shows a lever on a proximal end handle for catheter steering;

FIG. 4 shows a complete assembly in a non-deflected state;

FIG. 5 shows the assembly of FIG. 4 in a deflected state;

FIG. 6 illustrates the device in a surgical context;

FIG. 7 is a flow chart illustrating a method of delivering a medication according to an embodiment of the invention; and

FIG. 8 is a flow chart according to another embodiment of the invention.

### DETAILED DESCRIPTION OF THE INVENTION

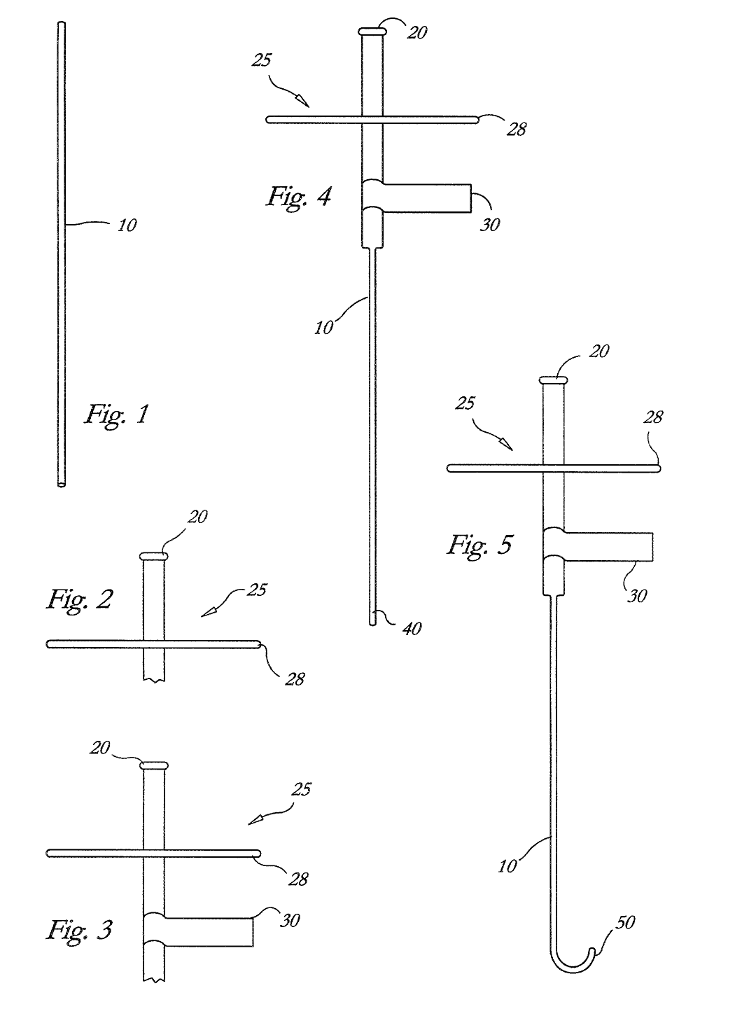

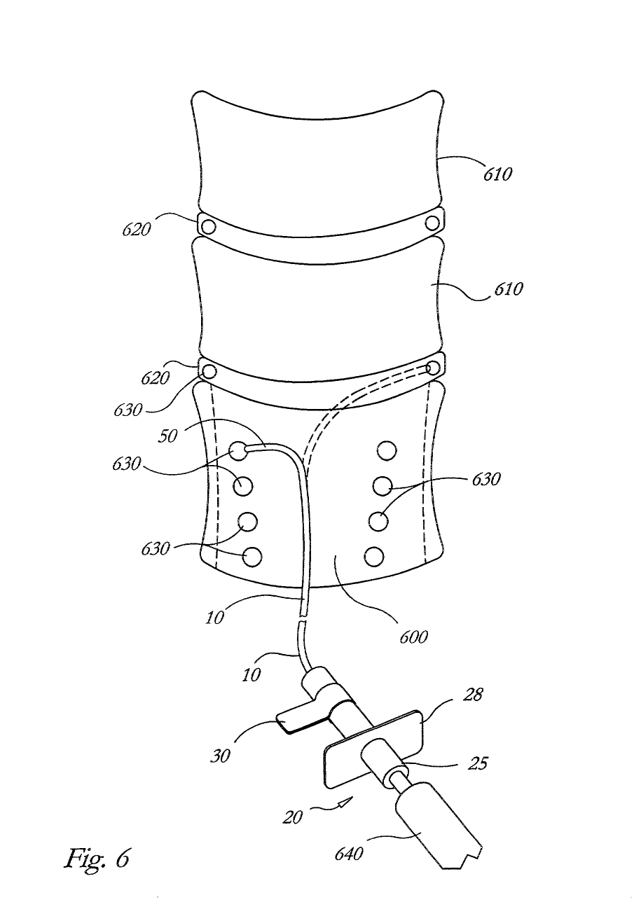

Referring to FIGS. 1 and 6, a steerable catheter body 10 is used to deliver medications to precisely selected locations 630 for the purpose of controlling pain.

FIGS. 2 and 3 illustrate an injection hub 25 used to control and manipulate catheter 10. Injection hub 25 includes an injection port 20, a handle 28 and a steering mechanism 30. Injection hub 25 is used to guide and control steerable catheter 10.

Now referring to FIGS. 4 and 5, catheter 10 is shown with a non-deflected tip 40, and with a deflected tip 50.

Now referring to FIG. 6, an operational diagram illustrating an embodiment of the present invention, steerable catheter 10 is inserted in epidural space 600 and carefully guided to precisely selected locations 630 to deliver pain medication. Deflected tip 50 is controlled using steering lever 30 to precisely position the end of catheter 10. Injector port 20 accepts a syringe 640 which contains the pain medication. The medication can be delivered between vertebrae 610 and intervertebral discs 620.

Although the kind of catheter shown is an example of a steerable catheter, it is understood that a practitioner skilled in the art could use any kind of catheter to practice the present invention as long as the catheter was steerable, enabling the practitioner to precisely deliver a medication to a selected location.

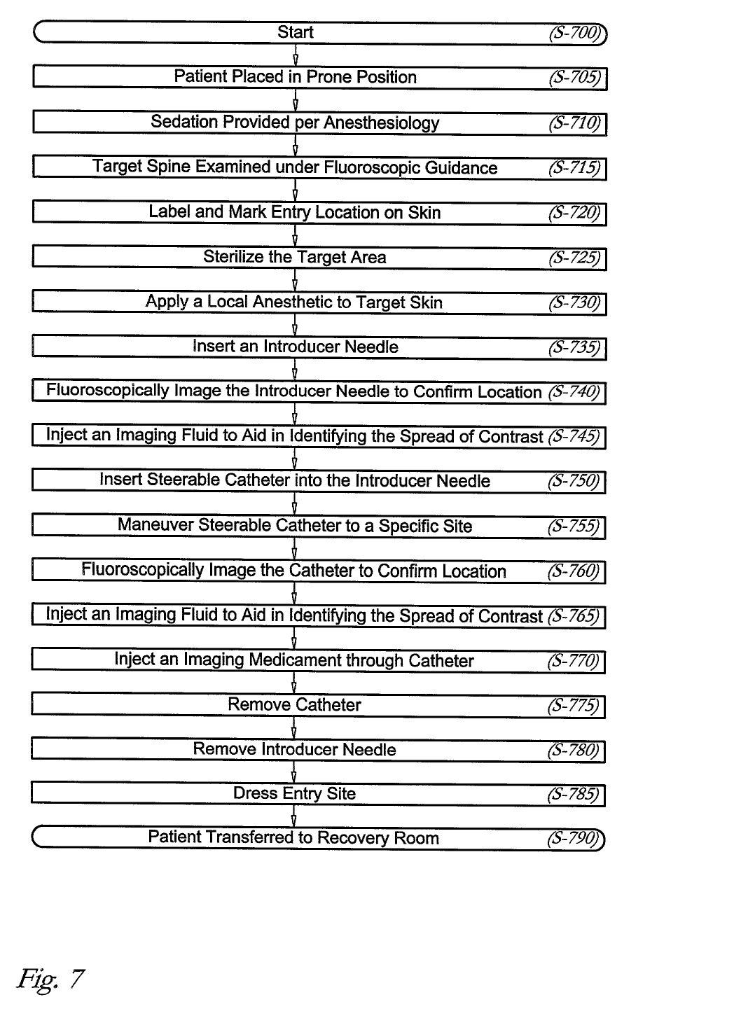

Referring now to FIG. 7, at the start of the procedure (S-700), the physician scrubs and generally prepares according to the norms of an operating room as is known in the art. The patient is preferably placed in a prone position (S-705). Standard operating procedures and routine monitoring of the patient are well known in the art and will not be described here. A sedative is administered to the patient by a qualified anesthesiologist (S-710).

In the next step, the physician examines the patient's spine (S-715). A fluoroscope is used to examine the spine, but other imaging devices may be used (i.e. CT scan) if appropriate. It is important that the physician be able to carefully select the target locations requiring medication in order for the present invention to be effective.

Utilizing the information from the fluoroscope, a point of entry in the cervical, thoracic, lumbar or sacral spine is determined and marked on the patient's skin (S-720).

The marked area is sterilized and a surgical drape is applied (S-725). Betadine® is commonly used to sterilize the skin prior to surgical procedures, but other sterilization agents would be acceptable to prepare the patient's skin.

To minimize discomfort to the patient, a topical anesthetic, such as Lidocaine 1%, is used to numb the entry location (S-730).

Once the patient's skin is sufficiently desensitized, an introducer needle is inserted through the skin at the marked point of entry (S-735). The size of the introducer needle must be of a sufficient diameter to accommodate a steerable catheter which will be inserted through the introducer needle. As an example, the present invention has been practiced utilizing a 16 gauge introducer needle. Of course, many size introducer needles are available commercially and could be used according to the method of the present invention, as long as the selected needle accommodates the steerable catheter chosen. Because of problems associated with penetrating the epidural space, the size of the introducer needle/steerable catheter should be chosen to minimize the size of the puncture.

In order to ensure that the medication is delivered to precisely the correct location, the introducer needle is imaged using a fluoroscope (S-740). Adjustments can be made at this time if the position of the introducer needle is not correct. An imaging fluid, such as Isovue M200®, is injected through the introducer needle so that the target area is able to be better imaged. The imaging fluid provides a good contrast spread making identification of the treatment area and location of the introducer needle easier to view.

Next a steerable catheter is inserted in through the introducer needle (S-750). The catheter can be of a type with a thumb or finger control to deflect the end of the catheter to aid the physician in delivering the medication to precise locations. Specific sites are selected to enhance the pain blocking ability of the medications (i.e. Marcaine®, Celestone® and Wydase®). Once the catheter enters the epidural space, the fluoroscope is again used to show the physician the position of the catheter and to carefully guide the catheter to a specific site such as a nerve root (S-755 and S-760). Again, as discussed above, an imaging fluid is used to enhance the contrast shown by the fluoroscope (S-765). By precisely positioning the tip of the catheter using a steerable catheter, the physician is easily able to select the exact spot to deliver the medication in order to maximize the effectiveness of the medication while minimizing potential side effects. Once the physician selects and positions the catheter at the precise location, a medication is injected to the selected site and is delivered through the catheter (S-770).

The catheter is then carefully removed to minimize the loss of spinal fluid (S-775). Once the catheter is removed, the introducer needle is also removed (S-780) and the wound is washed with a topical agent such as alcohol and a sterile dressing is applied (S-785).

The patient is transferred to the recovery room and carefully monitored (S-790).

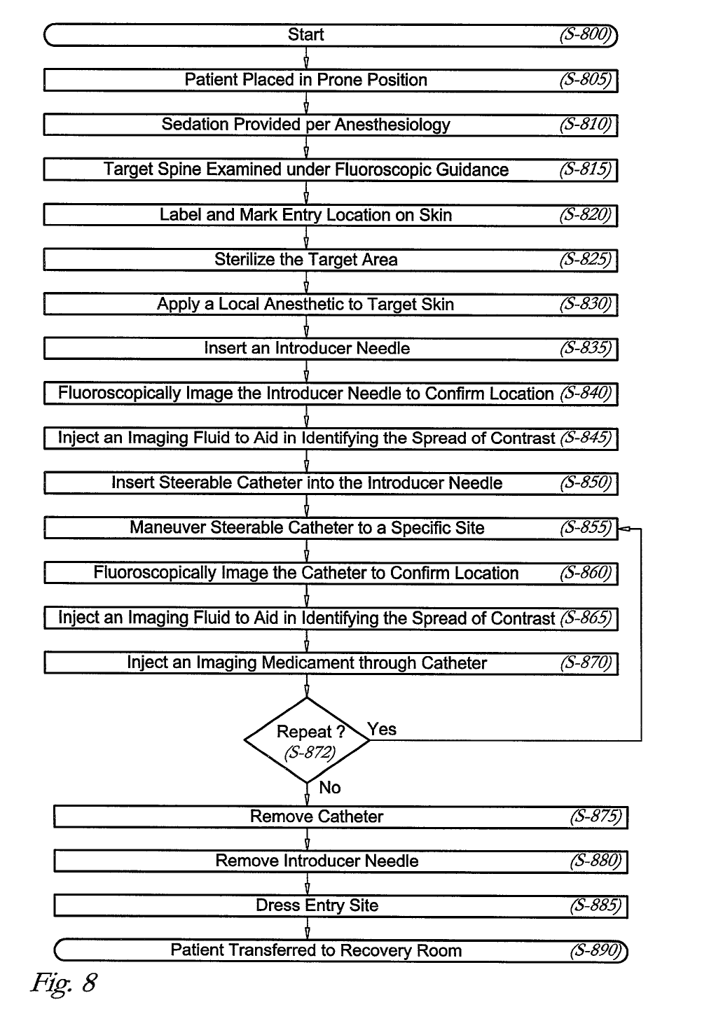

Referring now to FIG. 8, a flow chart illustrating another embodiment. The procedure outlined in FIG. 8 will be discussed only where it differs from the procedure outlined in FIG. 7.

In this embodiment, the physician is able to select multiple sites for treatment without moving the introducer needle. In Step S-855, the steerable catheter is positioned to a first location as determined by the fluoroscope as discussed above (S-860 and S-865) and a medication is delivered to that specific site (S-870).

However, utilizing the fluoroscope, the physician selects another site to deliver medication (S-872) and repeats the procedure as many times as necessary to further enhance the effectiveness of the pain controlling effect of the medications.

Additionally, the treatment may take place over an extended period of time (i.e. several days) following removal of the introducer needle and steering mechanism. If the physician desires to administer further medication over a prolonged period, the catheter can be secured to the patient's back with medical tape and medication can be delivered at a later time or via continuous infusion.

In the embodiment of the present invention where the treatment is prolonged, the catheter remains in place as discussed above, however, steering mechanism 30 and handle 28 are removed from catheter 10 in order to more comfortably remain attached to the patient. Although removing the steering mechanism will not allow the catheter to be further deflected, medication is delivered in the general area and continues to be effective for pain relief after selected sites have been targeted. Additionally, if further relocation of the catheter tip is required, steering mechanism 30 and handle 28 can be reinserted into catheter 10 for further manipulation of the catheter tip.

Once the physician determines that no more medication is needed in the single shot method, the catheter and introducer needle are removed (S-875 and 880) and the wound is cleansed and dressed (S-885) as above. The patient is then transferred to a recovery room (S-890).

Case Studies

Case 1: Intravenous line was started prior to entry into the operating room. Upon entry into the operating room, the patient was placed in a prone position. Routine monitoring protocols. Sedation was provided per anesthesiology. Next, the thoracic spine was examined under fluoroscopic guidance and the skin overlying the T10-T11 thoracic epidural space was labeled and marked. This was followed by sterile prep and drape of the thoracic spine with Betadine® solution. Next, the skin overlying the previously marked area was topicalized with Lidocaine 1% using a 25 gauge 1.5″ needle; approximately 5 cc's was used. Next, a 16 gauge introducer needle was placed in the right paramedian orientation using a translaminar approach and using a loss of resistance technique, the introducer needle was placed into the epidural space. Next, Isovue M200® (3 cc's were injected), which revealed a good spread of contrast in the epidural space. This confirmed AP and lateral views. Next, a directional epidural catheter was placed into the epidural space and easily steered into the right side of the thoracic epidural space using the hand-held steering device at the proximal end of the catheter. The tip was deflected posteriorly as well as to the right lateral side of the thoracic epidural space. The catheter tip was then straightened and slowly advanced selectively into the right side of the thoracic epidural space. The catheter tip was advanced to approximately the T8 thoracic intervertebral space where the patient has known metastasis to the vertebral body and ribs. Next, 2 cc's of Isovue M200® were injected through the injection port, once again confirming good spread of contrast within the posterior aspect of the epidural space. This confirmed accurate placement of the catheter into the thoracic epidural space.

Next, Bupivacaine 0.25% (3 cc's) was injected for anesthetic purposes, followed by the administration of Phenol 7.5% (approximately 3 cc's). Careful aspiration was performed throughout the procedure to make sure that there was no return of CSF or blood. At this point, after the Phenol 7.5% was administered, the catheter was taped to the back and left in situ for repeat injections through the catheter over the next several days for adequate neurolysis, enabling the patient to experience good relief of the right-sided chest wall pain. Next, the patient was transferred to the recovery room in supine position in stable condition, neurologically intact. The patient tolerated the procedure well.

Case 2: Intravenous line was started prior to entry into the operating room. Upon entry into the operating room, the patient was placed in a prone position. Routine monitoring protocols. Sedatives were administered to the patient with careful hemodynamic monitoring throughout the procedure. Next, the lumbosacral spine was examined under fluoroscopic guidance. The skin overlying the sacral hiatus was labeled and marked. This was followed by sterile prep and drape of the lumbosacral spine with Betadine® solution. Next, the skin overlying the sacrococcygeal ligament was topicalized with Lidocaine 1% using a 25 gauge 1.5″ needle, approximately 3 cc's were used. After this, a 16 gauge introducer needle was placed through the sacrococcygeal ligament with a slight leftward deflection for placement of the needle into the caudal epidural space. After this, Isovue M200® (approximately 2 cc's) was injected through the needle to confirm that the needle was in fact within the caudal epidural space. Next, a steerable epidural catheter was placed through the needle and directed with AP and lateral deflection to the left side of the lumbar epidural space. The catheter was brought up to the L4 neural foramen level. This was confirmed in AP and lateral views. Next, Isovue M200® (approximately 1 cc) was injected through the catheter, which indicated excellent outlining of the left L4 nerve root. After this, Bupivacaine 0.25% together with Celestone® (1 mg) was injected through the catheter for selective nerve root blockade at the L4 nerve root level on the left. The catheter was then withdrawn slightly and advanced more medially within the left side of the lumbar epidural space in a slightly cephalad orientation. The catheter tip was taken up to approximately L3. Next, Isovue M200® (1 cc) was injected, which revealed good spread of contrast selectively within the left side of the lumbar epidural space. Bupivacaine 0.25% (1 cc) together with Celestone® (1 mg) was injected at this level as well. Next, the catheter was withdrawn to the L5 neural foramen level and using the steerable qualities of the catheter, the catheter was directed into the left L5 neural foramen. The catheter was also advanced through the foramen along the left L5 nerve root. While the catheter was alongside the nerve root, Isovue M200® (1 cc) was injected, which revealed excellent spread of contrast around the left L5 nerve root, followed by administration of Bupivacaine 0.25% (1 cc) together with Celestone® (1 mg). Next, the catheter was withdrawn back more medially within the lumbar epidural space and Isovue M200® (1 cc) was injected, which once again demonstrated good spread of contrast within the left side of the lumbar epidural space. Bupivacaine 0.25% (1 cc) together with Celestone® (1 mg) was injected at this level. After this, the catheter was withdrawn through the introducer needle intact. All administration of medications was made through the injection port of the steerable/directional catheter. The introducer needle was removed, the sacrum was dressed with a bandage. The patient was next transferred to the recovery room in a supine position in stable condition, neurologically intact. The patient tolerated the procedure well.

Case 3: Intravenous line was started prior to entry into the operating room. Upon entry into the operating room, the patient was placed in a prone position. Routine monitoring protocols. Sedation was administered as seemed appropriate by the anesthesiologist. Next, the lumbosacral spine was examined under fluoroscopic guidance and the sacrum was also examined with fluoroscopic guidance using a lateral approach. Next, the skin over the sacral hiatus was labeled and marked, followed by sterile prep and drape of the lumbosacral spine with Betadine® solution. Next, the skin overlying the sacrococcygeal ligament was topicalized with Lidocaine 1% using a 25 gauge 1.5″ needle; approximately 10 cc's were administered. Next, a 16 gauge introducer needle was placed through the sacrococcygeal ligament and into the caudal epidural space. This was confirmed in a lateral fluoroscopic image and the needle was found to be well within the caudal canal. Next, Isovue M200® (5 cc's) was injected, which revealed excellent spread of contrast within the posterior aspect of the epidural space. Contrast was also noted around the sacral and lumbar nerve root. Using an AP fluoroscopic image, filling defect was noted in the right L5 through S2 lumbosacral nerve roots on the right.

After this, an epidural directional catheter was placed through the introducer needle and brought up to the level of the right L5 neural foramen. Using the thumb flex mechanical steering device, the catheter was easily placed off to the right. Prior to using the steerable device, there was difficulty placing the catheter within the posterior aspect of the epidural space, specifically enabling the catheter to exit through the right neural foramen along the right L5 nerve root. The directional catheter allowed excellent positioning of the tip of the catheter into the above-mentioned location. After this, Marcaine® 0.25% (2 cc's) together with Celestone® (2 mg) and Wydase® (300 units) were injected through the catheter. The catheter was withdrawn to the S1 and S2 foramina and the catheter was deflected in a posterior direction enabling good tip position at the right S1 and S2 levels. Next, Isovue M200® (approximately 8 cc's) was injected through the catheter in the right side of the lumbosacral epidural space and in those areas which previously showed poor flow of contrast, there was superior flow of contrast, which now indicated improvement of the previously noted filling defect at the L5 through S2 nerve root levels on the right side of the epidural space.

At this point, due to the fact that improved filling was noted at these levels, indicating repair of previous filling defect most likely due to epidural scar tissue, sodium chloride 10% (approximately 10 cc's) was injected in divided doses slowly, over approximately ten minutes to further cause dehydration and lysis of epidural adhesions as well as provide for good anti-inflammatory activity around the lumbosacral nerve roots at the above-mentioned level. Prior to injecting the saline, Bupivacaine 0.25% (approximately 10 cc's) was injected through the injection port of the catheter for topicalization of the epidural space to minimize the amount of discomfort usually produced by the injection of hypertonic saline into the epidural space. Lateral fluoroscopic image was taken which revealed that the catheter was in fact in the posterior aspect of the lumbar epidural space, also indicating once again that there was superior spread of contrast in the areas of the previously noted filling defect.

Careful aspiration was performed throughout the procedure to make sure that there was no return of cerebrospinal fluid or blood. Upon completion of injection of the medications, the catheter was removed intact followed by removal of the introducer needle. The lumbosacral spine was washed with alcohol, a sterile dressing was applied and the patient was next transferred to the recover room in a supine position in stable condition, neurologically intact. The patient tolerated the procedure well.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of delivering a beneficial agent to a patient employing a steerable catheter comprising the steps of:
positioning the steerable catheter within a selected target area of the patient to be treated;
imaging the steerable catheter wherein an operator is able to precisely maneuver the catheter to a specific location within the patient; and
injecting the beneficial agent at the specific location through the steerable catheter.

2. A method of delivering a beneficial agent to a patient according to claim 1, wherein the beneficial agent contains a pharmaceutically active agent.

3. A method of delivering a beneficial agent to a patient according to claim 1, wherein the steerable catheter includes a hand-held mechanical device for controlling the deflection of the steerable catheter.

4. A method of delivering a beneficial agent to a patient according to claim 1, wherein the imaging step includes a fluoroscopic device.

5. A method of delivering a beneficial agent to a patient according to claim 1, wherein the fluoroscopic device is used to guide the steerable catheter.

6. A method of delivering a beneficial agent to a patient according to claim 1, further including the steps of:
inserting an introducer needle into the selected target area of the patient for aligning the steerable catheter; and
inserting the steerable catheter into the introducer needle.

7. A method of delivering a beneficial agent to a patient according to claim 1, wherein the selected target area of the patient is the caudal epidural space.

8. A method of delivering a beneficial agent to a patient according to claim 1, wherein the steerable catheter is secured to the patient's back with medical tape.

9. A method of delivering a beneficial agent to a patient according to claim 8, wherein the beneficial agent is administered in discrete doses over a plurality of days.

10. A method of delivering medications employing a steerable catheter comprising the steps of:
providing a steerable catheter capable of delivering a medication to a selected area of a patient;
placing the patient in a prone position;
examining a target area of the patient;
marking an entry location within the target area;
sterilizing the target area;
inserting an introducer needle into the target area at the entry location;
confirming that the introducer needle is correctly positioned within the target area;
inserting the steerable catheter into the introducer needle;
maneuvering the steerable catheter to a precisely determined location within the target area;
confirming that the steerable catheter is correctly positioned at the precisely determined location; and
injecting the medication through the steerable catheter wherein the medication is delivered to the precisely determined location.

11. A method of delivering a beneficial agent to a patient according to claim 10, further comprising the step of:
securing the steerable catheter to the patient wherein further medications can be delivered without further manipulation of the steerable catheter.

12. A method of delivering a beneficial agent to a patient according to claim 10, wherein the steps of confirming that the introducer needle and the steerable catheter are correctly located include injecting a fluid to measure a spread of contrast.

13. A method of delivering a beneficial agent to a patient according to claim 10, further comprising the step of applying a local anesthetic in the target area to reduce the pain associated with inserting the introducer needle into the patient.

14. A method of delivering a beneficial agent to a patient according to claim 10, further comprising the steps of:
selecting at least one other location to be medicated;
maneuvering the steerable catheter to the at least one other location;
confirming that the steerable catheter is correctly positioned at the at least one other location; and
injecting the medication through the steerable catheter wherein the medication is delivered to the at least one other location.

15. A method of delivering a beneficial agent to a patient according to claim 8, wherein the steerable catheter includes a removable steering mechanism.

16. A method of delivering a beneficial agent to a patient according to claim 15, further comprising the step of removing the steering mechanism prior to securing the catheter to the patient's back with medical tape.
